(19) United States
(12) Patent Application Publication
(10) Pub. No.: US 2023/0028410 A1
(43) Pub. Date: Jan. 26, 2023

(54) PUMPLESS INTERNAL CIRCULATION PHOTOBIOREACTOR FOR HYDROGEN PRODUCTION

(71) Applicant: Henan Agricultural University, Zhengzhou (CN)

(72) Inventors: Zhiping ZHANG, Zhengzhou (CN); Quanguo ZHANG, Zhengzhou (CN); Yameng LI, Zhengzhou (CN); Huan ZHANG, Zhengzhou (CN); Bing HU, Zhengzhou (CN); Yang ZHANG, Zhengzhou (CN); Haorui ZHANG, Zhengzhou (CN)

(21) Appl. No.: 17/956,397

(22) Filed: Sep. 29, 2022

Related U.S. Application Data
(63) Continuation of application No. PCT/CN2020/142572, filed on Dec. 31, 2020.

(30) Foreign Application Priority Data
Nov. 9, 2020 (CN) 202011237409.7

(51) Int. Cl. C12M 1/00; C12M 1/12; C12M 1/107

(52) U.S. Cl. CPC C12M 21/02; C12M 23/06; C12M 23/22; C12M 23/36; C12M 23/38

(57) ABSTRACT

A new pumpless internal circulation photobioreactor for hydrogen production, including: an outer reaction barrel and an inner reaction barrel made of transparent materials, a gas collecting device, and an air duct arranged between the inner reaction barrel and the outer reaction barrel. An outer ring-shaped reaction chamber is formed between the inner reaction barrel and the outer reaction barrel. Liquid permeation holes and air holes are arranged on the inner reaction barrel. The gas collecting device is communicated with the outer ring-shaped reaction chamber. The air duct is communicated with the interior of the inner reaction barrel and the outer ring-shaped reaction chamber. The present invention has a simple and compact structure and is easy to operate, and gas produced by the reactor can be re-introduced into the reaction solution to provide aerodynamic power for stirring the reaction solution, which realizes low energy consumption and high-efficiency hydrogen production.

### CROSS-REFERENCE TO RELATED APPLICATIONS

This application is a continuation of International Application No. PCT/CN2020/142572 with a filling date of Dec. 31, 2020, designating the United States, now pending, and further claims to the benefit of priority from Chinese Application No. 202011237409.7 with a filing date of Nov. 9, 2020. The content of the aforementioned applications, including any intervening amendments thereto, are incorporated herein by reference.

### TECHNICAL FIELD

The present invention belongs to the technical field of rural energy in agricultural engineering, in particular to a new pumpless internal circulation photobioreactor for hydrogen production.

### BACKGROUND

With the rapid development of economy, a global demand for energy is constantly increasing, especially a demand for primary energy, which brings serious energy problems and environmental problems. Therefore, it is an urgent task to find a clean alternative energy.

In many new energy sources, hydrogen energy is an ideal alternative. A product of hydrogen after combustion or catalytic oxidation is water or water vapor, without greenhouse gases such as CO₂ or other pollutants, which can help to slow down the greenhouse effect and prevent environmental pollution. Hydrogen has a high calorific value and can replace fossil fuels such as coal, oil, and natural gas.

At present, conventional hydrogen production methods include physicochemical and biological methods.

Hydrogen production by the physicochemical method is at the expense of a large amount of fossil energy, which consumes energy and causes environmental pollution. Therefore, more and more researches begin to approach biohydrogen production.

The biohydrogen production has no shortcomings of the conventional hydrogen production, which has low costs and mild reaction conditions, and can achieve zero emission and no pollution.

Among many biohydrogen production methods, hydrogen production by photosynthesis has received extensive attention. The hydrogen production by photosynthesis is to use photosynthetic bacteria to convert organic matter into hydrogen by light under anaerobic conditions. No oxygen is produced in the whole process, and there is no inhibition effect of oxygen. Moreover, the photosynthetic bacteria grow fast, and a wide range of substrates are available. Common agricultural wastes and organic wastewater in daily life can be used as the substrates, and waste is also recycled during hydrogen production. Therefore, there is a tremendous need to research and develop a photobioreactor for hydrogen production, which is a key link of hydrogen production by photosynthesis from a laboratory scale to large-scale industrial production.

### SUMMARY OF THE INVENTION

In order to solve the shortcomings in the related art, the present invention provides a new pumpless internal circulation photobioreactor for hydrogen production with a compact structure, a small volume, convenient operation, high hydrogen production efficiency, and better energy conservation.

The present invention adopts the following technical solution.

A new pumpless internal circulation photobioreactor for hydrogen production, including:

an outer reaction barrel, an inner reaction barrel, a gas collecting device, and an air duct arranged between the inner reaction barrel and the outer reaction barrel;

the top of the outer reaction barrel being provided with a barrel cover, the outer reaction barrel, the inner reaction barrel, and the barrel cover being all made of transparent materials, the top of the outer reaction barrel being open, the barrel cover covering the top of the outer reaction barrel, and the top of the barrel cover being provided with an exhaust port;

the inner reaction barrel being arranged in the outer reaction barrel and the bottom of the inner reaction barrel being open, a bottom edge of the inner reaction barrel being in contact with the bottom of the outer reaction barrel, an outer ring-shaped reaction chamber being formed between the inner reaction barrel and the outer reaction barrel, interior of the inner reaction barrel being an inner circular reaction chamber, the bottom of the inner reaction barrel being provided with liquid permeation holes, and two groups of air holes being arranged on side walls of the inner reaction barrel;

the gas collecting device including a gas collecting bag, and the gas collecting bag being provided with a gas collecting pipe, the gas collecting pipe being connected to the exhaust port through a quick connector; and

two air ducts being arranged in the outer ring-shaped reaction chamber, and each air duct being communicated with the top of the inner reaction barrel and the bottom of the outer ring-shaped reaction chamber, wherein lower-end air outlets of the two air ducts are respectively arranged on two sides of the inner reaction barrel.

Further, at least three limit blocks are fixed to the bottom of the outer reaction barrel, all the limit blocks being in contact with an outer side wall of the inner reaction barrel.

Further, the gas collecting pipe is provided with a drying device and a valve, the valve being located between the drying device and the gas collecting bag.

Specifically, the new pumpless internal circulation photobioreactor for hydrogen production according to the present invention is used based on the following principle.

The barrel cover is removed, a reaction solution for hydrogen production is added to the outer reaction barrel, the reaction solution is adjusted to a condition suitable for growth and metabolism of photosynthetic bacteria, and a reaction substrate and photosynthetic bacteria are added for hydrogen production, which is a conventional hydrogen production reaction.

The inner circular reaction chamber in the inner reaction barrel is connected to the outer ring-shaped reaction chamber through the liquid permeation holes, so that the reaction solution can enter the inner circular reaction chamber through the liquid permeation holes.

Hydrogen produced by the reaction solution in the inner circular reaction chamber goes back below a liquid level of the reaction solution in the outer ring-shaped reaction chamber from the air duct down and stirs the reaction solution. In this way, on the one hand, mixing of the photosynthetic bacteria with the substrate can be promoted, a substrate conversion rate is increased, and more hydrogen gas is produced to overflow from the reaction solution. On the other hand, liquid in the outer ring-shaped reaction chamber is more turbid than that in the inner circular reaction chamber, making light transmittance of the exterior worse than that of the interior, which just meets a realistic environmental condition. That is, the exterior is at a close distance from a light source, and illumination intensity is high, which can enhance performance of hydrogen production. A small amount of light enters the inner circular reaction chamber through the outer ring-shaped reaction chamber, so that the reaction solution in the inner circular reaction chamber can also produce hydrogen.

In the process, the hydrogen overflowing from the reaction solution in the outer ring-shaped reaction chamber slowly rises above the liquid level of the reaction solution in the outer ring-shaped reaction chamber, then enters the gas collecting pipe through the exhaust port, and finally flows into the gas collecting bag through the gas collecting pipe.

Based on the above, the present invention has the following beneficial effects.

The present invention has a simple and compact structure and is easy to operate. On the basis of an existing internal circulation reactor, in the present invention, an original power device is changed and the reactor is designed to be cylindrical, which has uniform light transmission and occupies a small area. Moreover, one inner reaction barrel is mounted inside the outer reaction barrel and two air ducts are added to re-introduce the hydrogen produced in the inner reaction barrel into the reaction solution to provide aerodynamic power for stirring the reaction solution, which replaces a power device pump providing aerodynamic power for the internal circulation reaction, effectively saves energy consumption, and realizes low energy consumption and high-efficiency hydrogen production.

### BRIEF DESCRIPTION OF DRAWINGS

FIG. 1 is a schematic structural diagram according to the present invention.

FIG. 2 is a schematic structural diagram according to the present invention.

FIG. 3 is a schematic structural diagram according to the present invention.

FIG. 4 is a sectional view taken along a direction A-A in FIG. 3.

### DETAILED DESCRIPTION OF PREFERRED EMBODIMENTS

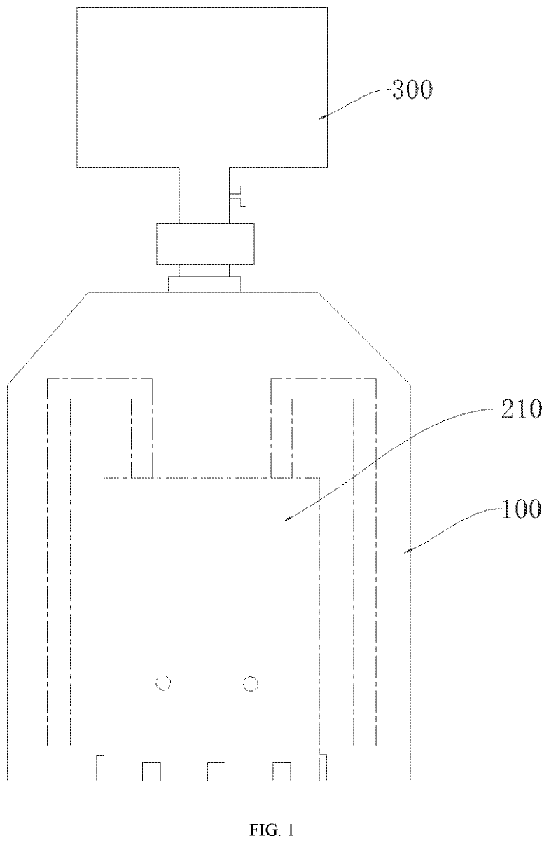

As shown in FIG. 1, a new pumpless internal circulation photobioreactor for hydrogen production according to the present invention includes: an outer reaction barrel 100, an inner reaction barrel 210, a gas collecting device 300, and an air duct 220 arranged between the inner reaction barrel 210 and the outer reaction barrel 100.

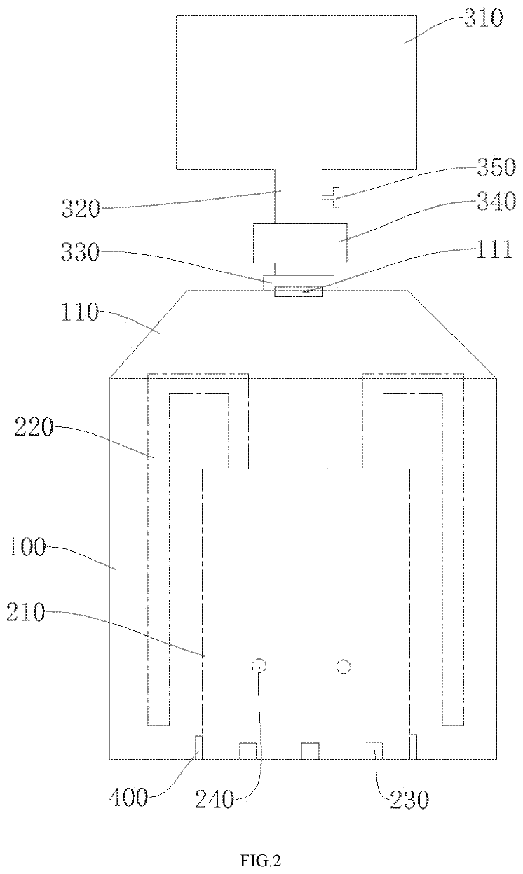

Referring to FIG. 2 to FIG. 4, specifically, the top of the outer reaction barrel 100 is provided with a barrel cover 110, the outer reaction barrel 100 and the barrel cover 110 are both made of transparent materials, the top of the outer reaction barrel 100 is open, the barrel cover 110 covers the top of the outer reaction barrel 100, and the top of the barrel cover 110 is provided with an exhaust port 111.

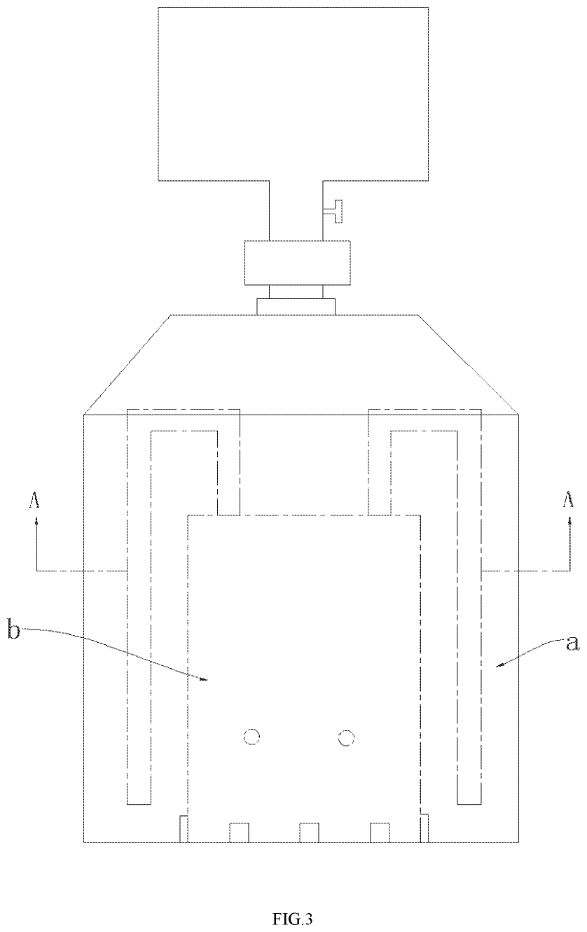

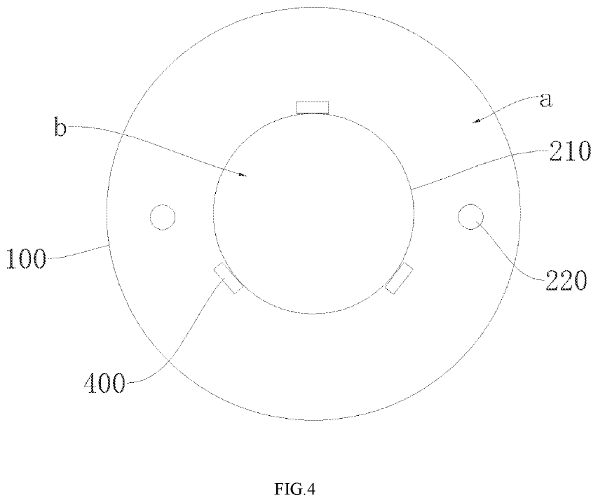

The inner reaction barrel 210 is made of a transparent material, which is arranged in the outer reaction barrel 100 and has an open bottom. A bottom edge of the inner reaction barrel 210 is in contact with the bottom of the outer reaction barrel 100. In this way, referring to FIG. 3 and FIG. 4, an outer ring-shaped reaction chamber a is formed between the inner reaction barrel 210 and the outer reaction barrel 100. Interior of the inner reaction barrel 210 is an inner circular reaction chamber b, and the bottom of the inner reaction barrel 210 is provided with liquid permeation holes 230. The inner circular reaction chamber b in the inner reaction barrel 210 is communicated with the outer ring-shaped reaction chamber a through the liquid permeation holes 230. In this way, a reaction solution can enter the inner circular reaction chamber b through the liquid permeation holes 230.

It may be understood that the outer reaction barrel 100, the barrel cover 110, and the inner reaction barrel 210 are all made of transparent materials (e.g., organic glass). In this way, the outer reaction barrel 100, the barrel cover 110, and the inner reaction barrel 210 have higher light transmissivity, which helps to absorb light therein. The outer reaction barrel 100 and the inner reaction barrel 210 are both cylindrical, which can effectively increase a specific surface area and improve uniformity of light transmission.

Referring to FIG. 2, the gas collecting device 300 includes a gas collecting bag 310, the gas collecting bag 310 is provided with a gas collecting pipe 320, and the gas collecting pipe 320 is connected to an exhaust port 111 through a quick connector 330. The arrangement of the quick connector 330 can facilitate the connection therebetween.

Referring to FIG. 2 to FIG. 4, two air ducts 220 are arranged in the outer ring-shaped reaction chamber a, and each air duct 220 is communicated with the top of the inner reaction barrel 210 and the bottom of the outer ring-shaped reaction chamber a. Lower-end air outlets of the two air ducts 220 are respectively arranged on two sides of the inner reaction barrel 210. With the arrangement of the air ducts 220, hydrogen produced in the inner circular reaction chamber b can flow into the outer ring-shaped reaction chamber a through the air ducts 220.

Specifically, the new pumpless internal circulation photobioreactor for hydrogen production according to the present invention is used based on the following principle.

Firstly, the barrel cover 110 is removed, and a reaction solution for hydrogen production is added to the outer reaction barrel 100. In the process, the inner circular reaction chamber b in the inner reaction barrel 210 is communicated with the outer ring-shaped reaction chamber a through the liquid permeation holes 230. Therefore, the reaction solution can flow into the inner circular reaction chamber b from the outer ring-shaped reaction chamber a through the liquid permeation holes 230.

At the same time, the reaction solution is adjusted to a condition suitable for growth and metabolism of photosynthetic bacteria, and then a reaction substrate is added to the outer ring-shaped reaction chamber a to enable the photosynthetic bacteria to produce hydrogen, which is a conventional hydrogen production reaction.

Both the outer reaction barrel 100 and the inner reaction barrel 210 have light transmittance. Therefore, the reaction solution in the inner circular reaction chamber b can also produce hydrogen. The hydrogen produced by the reaction solution in the inner circular reaction chamber b flows below a liquid level of the reaction solution in the outer ring-shaped reaction chamber a through the air duct 220 and stirs the reaction solution. In this way,

On the one hand, mixing of the photosynthetic bacteria with the substrate can be promoted, a substrate conversion rate is increased, and more hydrogen gas is produced. The hydrogen produced overflows from the reaction solution.

On the other hand, liquid in the outer ring-shaped reaction chamber a is more turbid than that in the inner circular reaction chamber b, making light transmittance in the outer ring-shaped reaction chamber a worse than that in the inner circular reaction chamber b, which just meets a realistic environmental condition. That is, the outer ring-shaped reaction chamber a is at a close distance from a light source, and illumination intensity is high, which can enhance efficiency of hydrogen production. A small amount of light enters the inner circular reaction chamber b through the outer ring-shaped reaction chamber a, so that the reaction solution in the inner circular reaction chamber b can also produce hydrogen.

In the above process of hydrogen production, the hydrogen overflowing from the reaction solution in the outer ring-shaped reaction chamber a slowly rises above the liquid level of the reaction solution in the outer ring-shaped reaction chamber a, then enters the gas collecting pipe 320 through the exhaust port 111, and finally flows into the gas collecting bag 310.

It may be understood that the process of hydrogen production is continuous in the present invention.

In addition, it is to be noted that, in the process of hydrogen production,

Firstly, the reaction solution is required not to be higher than the top of the inner circular reaction chamber b. In this way, a pipe orifice of the air duct 220 on the top of the inner circular reaction chamber b may not be sealed by the reaction solution, so that the hydrogen produced by the reaction solution can enter the air duct 220 smoothly and then flow downward to the bottom of the reaction solution to form a good stirring effect.

Secondly, the liquid level of the reaction solution is required to be separated from the top of the outer reaction barrel 100 by a certain space, which is called a gas-liquid separation region. Gas and liquid are required to be separated in this region to prevent entry of excessive liquid into the gas collecting bag 310.

Further, referring to FIG. 2, two groups of air holes 240 are arranged on side walls of the inner reaction barrel 210. In this way, the hydrogen produced in the inner circular reaction chamber b can flow into the outer ring-shaped reaction chamber a from the air ducts 220, and can also flow into the outer ring-shaped reaction chamber a through the air holes 240, so as to enhance the stirring effect on the reaction solution in the outer ring-shaped reaction chamber a.

Further, the two groups of air holes 240 are arranged on two opposite sides of the inner reaction barrel 210 respectively. At the same time, lower-end air outlets of the two air ducts 220 are respectively arranged on the two opposite sides of the inner reaction barrel 210. Moreover, the air holes 240 and the lower-end air outlets of the air ducts 220 are spaced along a circumference direction. In this way, hydrogen flowing through the air holes 240 and the lower-end outlets of the air ducts 220 can stir the reaction solution in the outer ring-shaped reaction chamber a evenly, so as to help to improve the stirring effect of the hydrogen flowing out of the air holes 240 and the air ducts 220 on the reaction solution.

Further, a filter screen is arranged at the middle of the liquid permeation holes 230 and the air holes 240 on the inner reaction barrel 210, so that entry of the reaction substrate in the outer ring-shaped reaction chamber a into the inner circular reaction chamber b can be prevented.

Further, at least three limit blocks 400 are fixed to the bottom of the outer reaction barrel 100, and all the limit blocks 400 are in contact with an outer side wall of the inner reaction barrel 210. The arrangement of the limit blocks 400 can ensure the position of the inner reaction barrel 210 in the outer reaction barrel 100 and ensure stability of a relative position between the inner reaction barrel 210 and the outer reaction barrel 100 during the hydrogen production.

Further, referring to FIG. 2, the gas collecting pipe 320 is provided with a drying device 340 and a valve 350, and the valve 350 is located between the drying device 340 and the gas collecting bag 310. On the one hand, the arrangement of the valve 350 can facilitate collection of the hydrogen during the action, and after the reaction, the valve 350 can be closed, so as to close and store the collected hydrogen in the gas collecting bag 310 to prevent leakage of hydrogen. On the other hand, the arrangement of the drying device 340 can adsorb water vapor carried in the hydrogen, so as to dry the hydrogen.

Based on the above, the present invention has the following beneficial effects.

Firstly, the inner reaction barrel 210 divides an inner space of the outer reaction barrel 100 into an outer high solid-phase region and an inner low solid-phase region, which solves common problems such as insufficient illumination inside the reactor.

Secondly, the hydrogen produced by the reaction solution in the inner reaction barrel 210 can flow into the outer reaction barrel 100, which provides stirring for the outer high solid-phase reaction solution, increases mass and light transfer, and promotes escape of gas phase products.

Thirdly, positions of the liquid permeation holes 230 on the inner reaction barrel 210 and the lower-end outlets of the air ducts 220 are arranged in a staggered manner to prevent flowing of turbulent flow of solid matter into the inner reaction barrel 210 caused by gas stirring.

What is claimed is:

1. A new pumpless internal circulation photobioreactor for hydrogen production, comprising:
an outer reaction barrel, an inner reaction barrel, a gas collecting device, and an air duct arranged between the inner reaction barrel and the outer reaction barrel;
the top of the outer reaction barrel being provided with a barrel cover, the outer reaction barrel, the inner reaction barrel, and the barrel cover being all made of transparent materials, the top of the outer reaction barrel being open, the barrel cover covering the top of the outer reaction barrel, and the top of the barrel cover being provided with an exhaust port;
the inner reaction barrel being arranged in the outer reaction barrel and the bottom of the inner reaction barrel being open, a bottom edge of the inner reaction barrel being in contact with the bottom of the outer reaction barrel, an outer ring-shaped reaction chamber being formed between the inner reaction barrel and the outer reaction barrel, interior of the inner reaction barrel being an inner circular reaction chamber, the bottom of the inner reaction barrel being provided with liquid permeation holes, and two groups of air holes being arranged on side walls of the inner reaction barrel;
the gas collecting device comprising a gas collecting bag, and the gas collecting bag being provided with a gas collecting pipe, the gas collecting pipe being connected to the exhaust port through a quick connector; and
two air ducts being arranged in the outer ring-shaped reaction chamber, and each air duct being communicated with the top of the inner reaction barrel and the bottom of the outer ring-shaped reaction chamber, wherein lower-end air outlets of the two air ducts are respectively arranged on two sides of the inner reaction barrel.

2. The new pumpless internal circulation photobioreactor for hydrogen production according to claim 1, wherein at least three limit blocks are fixed to the bottom of the outer reaction barrel, all the limit blocks being in contact with an outer side wall of the inner reaction barrel.

3. The new pumpless internal circulation photobioreactor for hydrogen production according to claim 2, wherein the gas collecting pipe is provided with a drying device and a valve, the valve being located between the drying device and the gas collecting bag.
